(19) United States
(12) Patent Application Publication
(10) Pub. No.: US 2018/0220714 A1
(43) Pub. Date: Aug. 9, 2018

(54) GARMENT FOR FOOT WITH TRIANGULAR ANKLE PANELS

(71) Applicant: NIKE, Inc., Beaverton, OR (US)

(72) Inventors: HANNAH R. AMIS, PORTLAND, OR (US); RONEN YEHUDA, SECAUCUS, NJ (US)

(21) Appl. No.: 15/425,475

(22) Filed: Feb. 6, 2017

(51) Int. Cl. A41B 11/00; A61F 13/08; A43B 17/00

(52) U.S. Cl. CPC A41B 11/007; A41B 11/003; A41B 2400/38; A43B 17/003; A61F 13/08

(57) ABSTRACT

A knit garment is provided to be worn on the foot that has a toe, foot, and heel area. The garment also has a medial triangular ankle panel and a lateral triangular ankle panel. The medial triangular ankle panel has a first medial vertex near the rear, top portion of the foot area, a second medial vertex near the top of the heel area, and a third medial vertex below and between the first and second medial vertices. The lateral triangular ankle panel has a first lateral vertex near the rear, top portion of the foot area, a second lateral vertex near the top of the heel area, and a third lateral vertex below and between the first and second lateral vertices. The sides of the medial and lateral triangular panels extending between the respective first and second medial and lateral vertices define an opening of the garment for a foot.

### TECHNICAL FIELD

The present disclosure relates to garments, such as socks or footies, designed to be worn on a foot. More particularly, the disclosure relates to a method of knitting such garments, and the garments themselves, that provide a better fit.

### BACKGROUND

Various styles of socks are now available. Commercially manufactured socks are typically produced using circular knitting machines. These machines employ needles mounted on a cylinder, or sometimes a double cylinder. The cylinder spins and the needles interlock loops of yarn. In some aspects, the circular knitting process results in a circular tube that is open on both ends. Additionally, different sock styles are now available, such as a calf-length sock, a mid-calf sock, or a footie designed to terminate around the ankle of the wearer. In a typical footie-type sock, the open end (allowing entry of the wearer's foot) is typically a generally circular opening, produced by the circular knitting process, having a finished, welted cuff. This opening may result in a fit that could be improved. The sock described below is manufactured on circular knitting machines with a structure providing a better fit around the ankle area. BRIEF SUMMARY

This Summary provides a high-level overview of the disclosure and introduces a selection of concepts that are further described in the Detailed Description below. This Summary is not intended to identify key features or essential features of the claimed subject matter, nor is it intended to be used as an aid in isolation to determine the scope of the claimed subject matter.

Aspects herein generally relate to a garment to be worn on the foot, such as a sock. The garment has a toe area, and a foot area having a top and a bottom, the foot area being contiguous with the toe area. The garment further has a heel area that is contiguous with at least a portion of the foot area. The garment has a medial triangular ankle panel and a lateral triangular ankle panel. The medial triangular ankle panel has a first medial vertex near the rear, top portion of the foot area, a second medial vertex near the top of the heel area, and a third medial vertex below the first and second medial vertices, generally midway between the first and second medial vertices. The lateral triangular ankle panel has a first lateral vertex near the rear, top portion of the foot area, a second lateral vertex near the top of the heel area, and a third lateral vertex below the first and second lateral vertices, generally midway between the first and second lateral vertices. The side of the medial triangular panel extends between the first and second medial vertices, and the side of the lateral triangular panel extends between the first and second lateral vertices, defining an opening of the garment for a foot. In some aspects, this opening is a non-circular opening, such as an oval.

In an additional aspect, a method of knitting a garment, using a circular knitting machine, to be worn on a foot, is provided. The method includes knitting a toe area and foot area using all of the needles on the circular knitting machine. The method further includes knitting a first triangular panel in a first ankle area, adjacent the foot area, using only a first portion of the needles on the circular knitting machine, while the remaining needles hold yarns from the last course of knitting the foot area. The method further includes knitting a second triangular panel in a second ankle area opposite from the first ankle area, using only a second portion of the needles on the circular knitting machine, while the remaining needles hold the yarn from the last course of knitting the first triangular panel. The method further includes resuming knitting of the garment using all of the needles of the circular knitting machine. The method could also be reversed, starting at a top of the sock, and knitting toward the toe (rather than starting the knitting with the toe).

In an additional aspect, a method of knitting a sock having a tubular structure with at least a closed toe end and an open end, using a circular knitting machine, is provided. The method includes knitting a first triangular panel at the open end, using only a first portion of the needles on the circular knitting machine, while the remaining needles hold yarns from the last course of knitting prior to knitting the first triangular panel. The method further includes knitting a second triangular panel at the open end, opposite from the first triangular panel, using only a second portion of the needles on the circular knitting machine, while the remaining needles hold the yarn from the last course of knitting the first triangular panel. In some aspects, the first and second triangular panels form a non-circular opening for the sock, such as an oval.

Additional objects, advantages, and novel features of the invention will be set forth in part in the description which follows, and in part will become apparent to those skilled in the art upon examination of the following, or may be learned by practice of the invention.

### BRIEF DESCRIPTION OF THE DRAWINGS

The present invention is described in detail below with reference to the attached drawing figures, which are incorporated herein by reference, wherein:

FIG. 1 is a side view of an exemplary left sock illustrating some aspects of the disclosure;

FIGS. 2A-2B show a top view of the sock of FIG. 1;

FIG. 3 is a diagrammatic representation of knitting a first triangular ankle panel;

FIG. 4 is a diagrammatic representation of knitting a second triangular ankle panel;

FIG. 5 is a diagrammatic representation of continued knitting after the first and second ankle panels;

FIG. 6 shows a portion of the garment, with overlaid lines showing the first triangular ankle panel;

FIG. 7 shows a portion of the garment, with overlaid lines showing the second triangular ankle panel; and

FIG. 8 is a diagram of an exemplary method of knitting a garment, according to aspects of this disclosure.

### DETAILED DESCRIPTION

The subject matter of aspects of the present invention is described with specificity herein to meet statutory requirements. But the description itself is not intended to necessarily limit the scope of the claims. Rather, the claimed subject matter might be embodied or carried out in other ways to include different elements or combinations of elements similar to the ones described in this document, in conjunction with other present or future technologies.

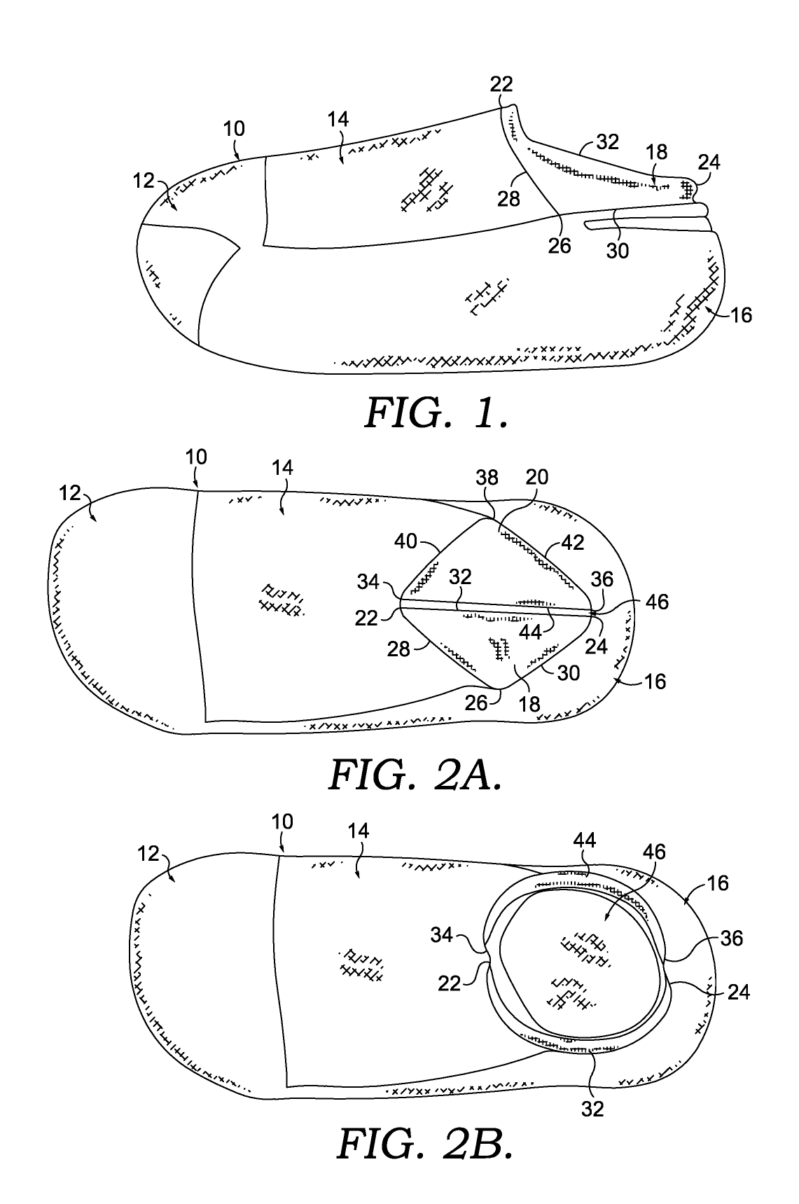

In general, this disclosure relates to a garment having features that provide a fit better adapted to a wearer's body. More specifically, the disclosure, in some aspects, relates to a sock having an opening better adapted to fit around the leg, or ankle area, of the wearer. Referring initially to FIG. 1, a sock 10 is depicted that is made using a circular knitting machine. The sock 10 shown in FIGS. 1 and 2A-2B is foot-agnostic, but could also be formed having features adapting the sock to be left, or right, foot specific. The sock 10 includes a closed toe area 12 contiguous with a foot area 14. As further detailed below, the sock 10 is typically knitted on a circular knitting machine, starting with toe area 12. The sock 10 could also be knitted starting at the other end of the sock 10, such as in a heel area 16. Heel area 16 is contiguous with a portion of the foot area 14 and is typically knit in a cup configuration in a form to fit a wearer's heel. A first triangular ankle panel 18 is knit, using methods detailed below, contiguous with an upper portion of the foot area 14 and heel area 16. Similarly, with reference to FIG. 2A, a second triangular ankle panel 20 is knit, using methods detailed below, contiguous with an upper portion of the foot area 14 and heel area 16, opposite first triangular panel 18. First triangular panel 18 may generally be seen to have three vertices. The first vertex 22 is near the top of the sock 10, at the rear of foot area 14. The second vertex 24 is near the top of the heel area 16. The third vertex 26 is located below, and generally midway between, the first vertex 22 and the second vertex 24. In some aspects, the side 28 extending between the first vertex 22 and the third vertex 26 extends as a boundary between the foot area 14 and the first triangular ankle panel 18. The side 30 extending between the second vertex 24 and the third vertex 26 extends as a boundary between the heel area 16 and the first triangular ankle panel 18. The top side 32 of first triangular ankle panel 18 extends between first vertex 22 and second vertex 24. In some aspects, top side 32 is knit as a non-welted, finished edge.

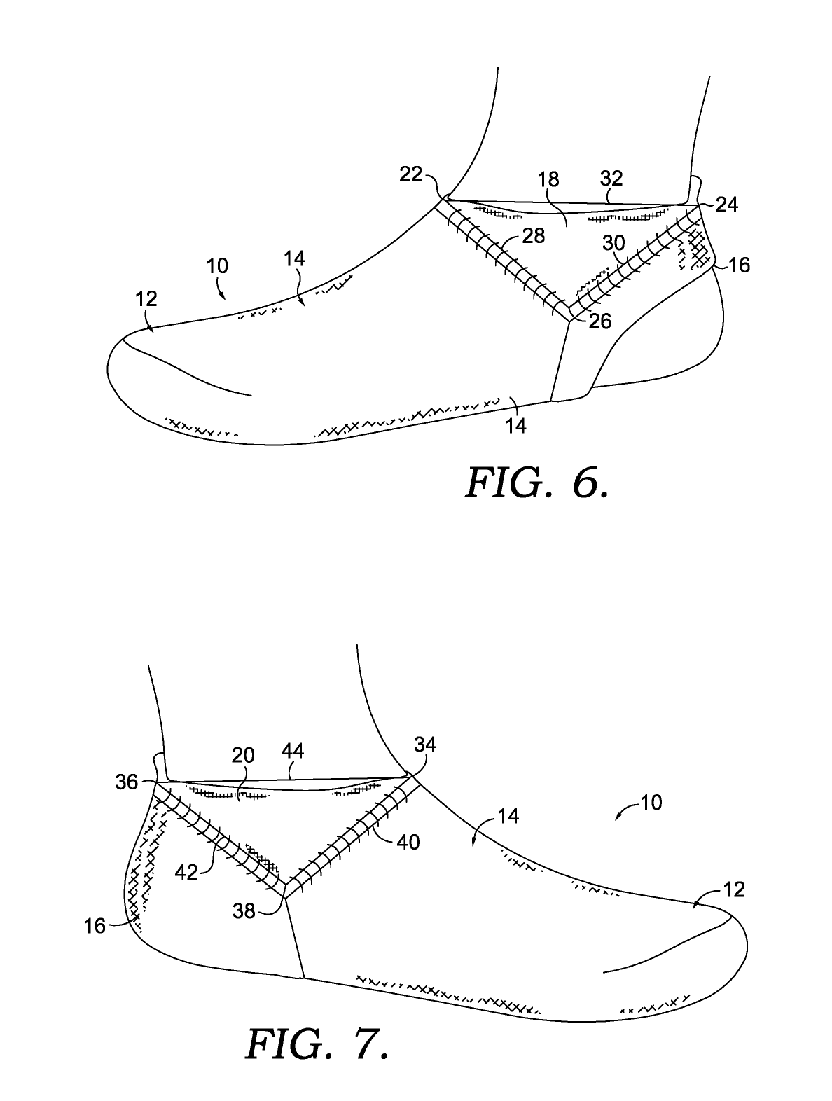

With reference to FIG. 2A, the second triangular ankle panel 20 may also generally be seen to have three vertices. The first vertex 34 is near the top of the sock 10, at the rear of foot area 14. The second vertex 36 is near the top of the heel area 16. The third vertex 38 is located below, and generally midway between, the first vertex 34 and the second vertex 36. In some aspects, the side 40 extending between the first vertex 34 and the third vertex 38 extends as a boundary between the foot area 14 and the second triangular ankle panel 20. The side 42 extending between the second vertex 36 and the third vertex 38 extends as a boundary between the heel area 16 and the first triangular ankle panel 18. The top side 44 of second triangular ankle panel 20 extends between first vertex 34 and second vertex 36. In some aspects, top side 44 is knit as a non-welted, finished edge. The triangular ankle panels 18 and 20 may be knit with an elastic yarn, and the elastic yarn may be more elastic than the yarns used to knit the remainder of sock 10. As best seen in FIGS. 6 and 7, the top sides 32 and 44 may tend to roll to some degree, due to the tension of the elastic yarns forming first and second triangular ankle panels 18 and 20, respectively. As best seen in FIG. 2B, the top sides 32 and 44 form a foot opening 46. FIG. 2A shows foot opening 46 as a generally linear gap between top side 32 and top side 44. In some aspects, if top side 32 and top side 44 are not held in an unrolled condition, the foot opening 46 expands (due to the configuration of first triangular ankle panel 18 and second triangular panel 20, and the elasticity of the yarns of panels 18 and 20) to form a non-circular foot opening 46. In one exemplary aspect, the foot opening 46 is a generally oval shape as shown in FIG. 2B.

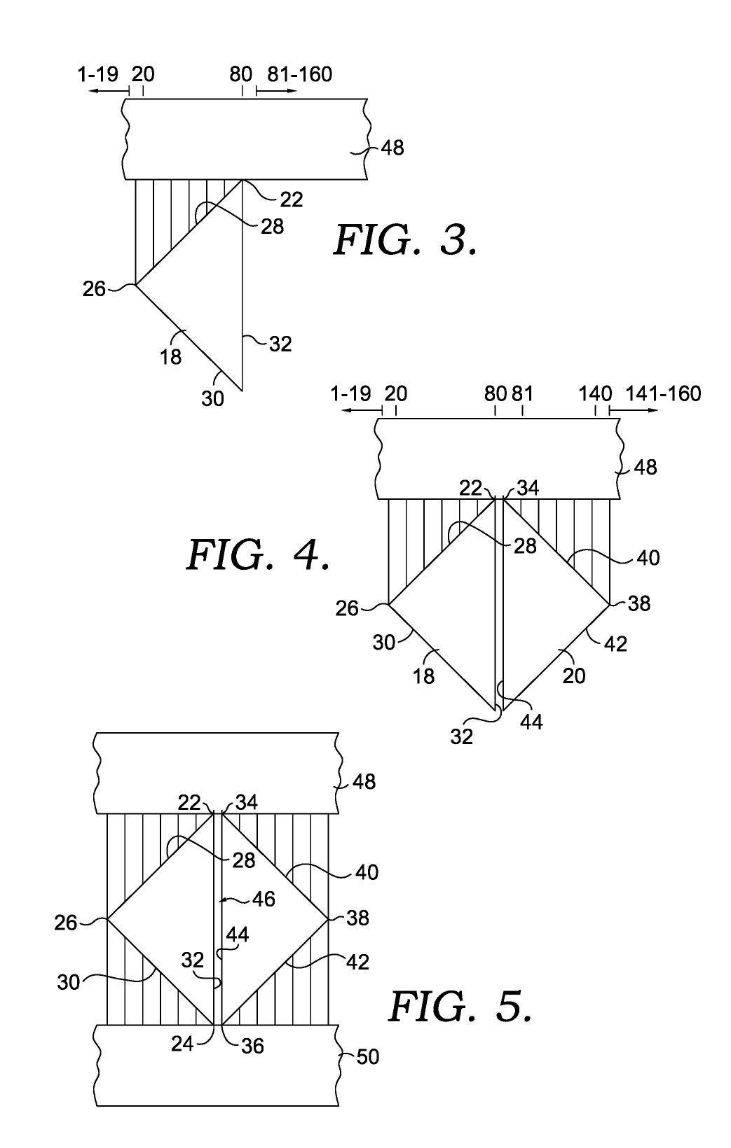

With reference to FIGS. 3-5, an exemplary aspect of knitting the first triangular panel 18 and the second triangular panel 20 is shown, somewhat schematically. The knitting of sock 10 typically starts with toe area 12 and moves to foot area 14. The knitting could also start with the heel area, in some aspects. To simplify the description of knitting the first and second triangular panels 18 and 20, with initial reference to FIG. 3, a band 48 is shown, representing a portion of the knitting of foot area 14 immediately prior to knitting first triangular ankle panel 18. Up to and including band 48, standard circular knitting techniques are used to knit toe area 12 and foot area 14, using all of the needles in the circular knitting machine. When the first triangular ankle panel 18 is to be knit, the circular knitting machine uses only a portion of the needles, while the remaining needles hold the yarns from the last course of standard circular knitting. In one exemplary aspect, on a circular knitting machine having one hundred sixty needles, first triangular ankle panel 18 is knit using only needles twenty through eighty in a reciprocating knitting motion between needles twenty through eighty. In this exemplary aspect, needle twenty corresponds to the location of third vertex 26, and needle eighty corresponds to the location of first vertex 22. The first knitting course between needles twenty and eighty thus forms side 28 of first triangular ankle panel 18. Needles one through nineteen and needles eighty-one through one hundred sixty hold the yarns from the last standard circular knitting of foot area 14, with the various numbered needles shown in FIG. 3. It should be understood that other circular knitting machines, having a different number of needles could be used, with comparable needle ratios. The knitting process continues as the first triangular panel 18 is knit with reciprocating knitting between needles twenty and eighty, forming the top side 32 and the side 30, as shown in FIG. 3.

After the first triangular ankle panel 18 is knit, the second triangular ankle panel 20 is knit, as shown in FIG. 4. To knit the second triangular ankle panel 20, again only a portion of the needles on the circular knitting machine are used. Continuing with the exemplary aspect above, on a circular knitting machine with one hundred sixty needles, only needles eighty-one through one hundred forty are used to knit second triangular ankle panel 20. The remaining needles hold the last course knit by the respective needles. The first course of knitting between needle eighty-one and one hundred forty form the side 40 between the first vertex 34 and the third vertex 38. The reciprocating knitting between needles eighty-one and one hundred forty form the top side 44 and side 42, as seen in FIG. 4. Once the second triangular ankle panel 20 is knit, standard circular knitting techniques are used, with all of the needles on the circular knitting machine knitting, starting from the last courses of the first triangular ankle panel 18 and the second triangular ankle panel 20, shown as a band 50 in FIG. 5. In an exemplary aspect, this standard circular knitting forms the connection of the first triangular ankle panel 18 and the second triangular ankle panel 20 to the heel area 16, along sides 30 and 42, as seen in FIG. 5. Note that in FIGS. 3-5, the connections along sides 28, 30, 40, and 42 are shown as long lines for the sake of clarity. In reality, these lines are just single stitches.

FIG. 6 shows a portion of the sock 10, showing the sides 28, 30, and 32 of the first triangular ankle panel 18 in solid, overlaid lines. In reality, lines representing sides 28, 30, and 32 are not as visible, but are shown here for clarity. As described above, the foot area 14 of sock 10 is knit with standard circular knitting techniques. First triangular ankle panel 18 is then knit as described above with respect to FIGS. 3 and 4. The heel area 16 shown in FIG. 6 is knit after the second triangular ankle panel 20 is knit. As shown in FIG. 6, the heel area 16 may be knit to form an open heel area, in some aspects. FIG. 7 is similar to FIG. 6, but shows second triangular ankle area 20 (and illustrates a closed heel area 16 aspect of the sock 10). The sides 40, 42, and 44 are shown in solid, overlaid lines, but in reality the lines representing these sides are not as visible. As described above, the foot area 14 of sock 10 is knit with standard circular knitting techniques, followed by the first triangular ankle panel 18 (FIG. 6) and then the second triangular ankle panel 20. After the second triangular ankle panel 20 is knit, the heel area 16 is knit with standard circular knitting techniques.

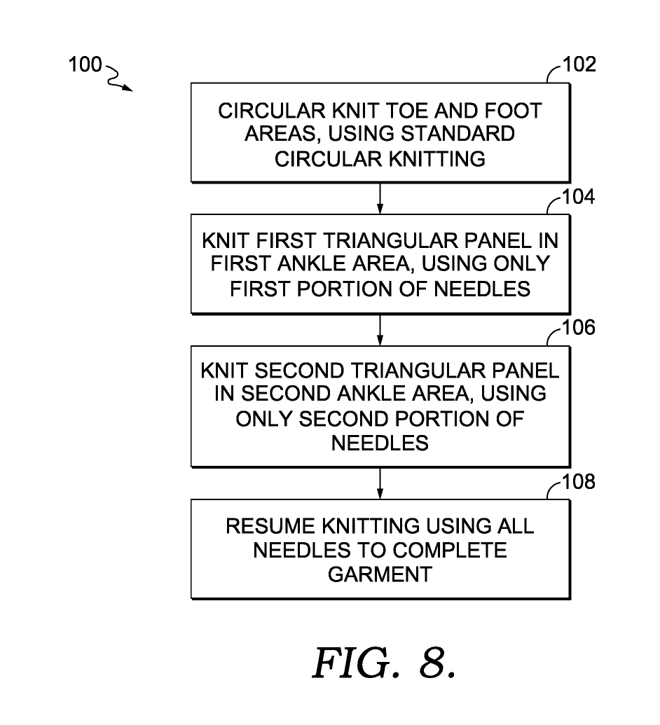

An exemplary method 100 of knitting the sock 10 is shown in FIG. 8. As shown at block 102, the toe and foot areas of the sock are knit using standard circular knitting techniques, typically using all of the needles in the circular knitting machine. After the foot area is knit, a first triangular ankle panel (such as first triangular ankle panel 18) is knit, as shown in FIG. 8 at block 104. In knitting the first triangular panel, the method includes programming the circular knitting machine to use only a portion of the needles, while the remaining needles hold the yarns from the last course of standard circular knitting. After the first triangular ankle panel is knit, the method continues at block 106 in knitting a second triangular ankle panel (such as second triangular ankle panel 20). In knitting the second triangular panel, the method includes programming the circular knitting machine to use only a portion of the needles, different from the needles used to knit the first triangular panel, while the remaining needles hold the yarns from the last course of knitting. Once the second triangular ankle panel is knit, standard circular knitting continues as shown at block 108, with all of the needles on the circular knitting machine knitting to finish the garment. As mentioned above, the method could be essentially reversed in some aspects, starting at the top, or the heel area, and knitting to the toe area.

From the foregoing, it will be seen that this invention is one well adapted to attain all the ends and objects hereinabove set forth together with other advantages which are obvious and which are inherent to the structure.

It will be understood that certain features and sub-combinations are of utility and may be employed without reference to other features and sub-combinations. This is contemplated by and is within the scope of the claims.

Since many possible embodiments may be made of the invention without departing from the scope thereof, it is to be understood that all matter herein set forth or shown in the accompanying drawings is to be interpreted as illustrative and not in a limiting sense.

1. A garment to be worn on the foot, comprising:
a toe area;
a foot area contiguous with the toe area, the foot area having a top and a bottom;
a heel area contiguous with at least a portion of the foot area, the heel area having a top and a bottom; and
a medial triangular ankle panel having a first medial vertex near the rear, top portion of the foot area, a second medial vertex near the top of the heel area, and a third medial vertex below the first and second medial vertices, generally midway between the first and second medial vertices; and
a lateral triangular ankle panel having a first lateral vertex near the rear, top portion of the foot area, a second lateral vertex near the top of the heel area, and a third lateral vertex below the first and second lateral vertices, generally midway between the first and second lateral vertices,
wherein the side of the medial triangular panel extending between the first and second medial vertices, and the side of the lateral triangular panel extending between the first and second lateral vertices define an opening of the garment for a foot.

2. The garment of claim 1, wherein the garment is a sock and the medial and lateral triangular panels are knitted with an elastic yarn.

3. The garment of claim 2, wherein the elastic yarn is a spandex yarn.

4. The garment of claim 1, wherein the opening defined by the side of the medial triangular panel extending between the first and second medial vertices, and the side of the lateral triangular panel extending between the first and second lateral vertices, is a non-circular opening.

5. The garment of claim 4, wherein the defined opening is an oval shape.

6. The garment of claim 5, wherein the side of the medial triangular panel extending between the first and second medial vertices, and the side of the lateral triangular panel extending between the first and second lateral vertices are non-welted.

7. A method of knitting a garment, using a circular knitting machine, to be worn on a foot, comprising:
knitting a toe area and a foot area using all of the needles on the circular knitting machine;
knitting a first triangular panel in a first ankle area, adjacent the foot area, using only a first portion of the needles on the circular knitting machine, while the remaining needles hold yarns from the last course of knitting the foot area;
knitting a second triangular panel in a second ankle area opposite from the first ankle area, using only a second portion of the needles on the circular knitting machine, while the remaining needles hold the yarn from the last course of knitting the first triangular panel; and
resuming knitting of the garment using all of the needles of the circular knitting machine.

8. The method of claim 7, wherein the first portion of the needles used to knit the first triangular panel is different from the second portion of the needles used to knit the second triangular panel.

9. The method of claim 8, further comprising using an elastic yarn to knit the first and second triangular panels.

10. The method of claim 9, wherein the first triangular panel has a free side adjacent to a free side of the second triangular panel, and wherein the free sides of the first and second triangular panels define an opening in the garment.

11. The method of claim 10, wherein the defined opening is non-circular.

12. The method of claim 11, further comprising knitting the free sides of the first and second triangular panels such that they are non-welted.

13. A method of knitting a sock having a tubular structure with at least a closed toe end and an open end, using a circular knitting machine, comprising:
knitting a first triangular panel at the open end, using only a first portion of the needles on the circular knitting machine, while the remaining needles hold yarns from the last course of knitting prior to knitting the first triangular panel;
knitting a second triangular panel at the open end, opposite from the first triangular panel, using only a second portion of the needles on the circular knitting machine, while the remaining needles hold the yarn from the last course of knitting the first triangular panel.

14. The method of claim 13, wherein the first portion of the needles used to knit the first triangular panel is different from the second portion of the needles used to knit the second triangular panel.

15. The method of claim 14, further comprising using an elastic yarn to knit the first and second triangular panels.

16. The method of claim 15, wherein the first triangular panel has a free side adjacent to a free side of the second triangular panel, and wherein the free sides of the first and second triangular panels define the opening of the open end.

17. The method of claim 16, wherein the defined opening is non-circular.

18. The method of claim 16, further comprising knitting the free sides of the first and second triangular panels such that they are non-welted.
